(19) United States
(12) Patent Application Publication
(10) Pub. No.: US 2024/0269671 A1
(43) Pub. Date: Aug. 15, 2024

(54) Airborne monitor to detect SARS-CoV-2 wild-type and mutations in airborne samples using Nucleic Acid Amplification Techniques (NAT)

(71) Applicant: THE BOARD OF TRUSTEES OF THE UNIVERSITY OF ILLINOIS, URBANA, IL (US)

(72) Inventors: Igor Paprotny, Chicago, IL (US); Michael S. Caffrey, Chicago, IL (US); Nitin S. Jayakumar, Chicago, IL (US)

(73) Assignee: THE BOARD OF TRUSTEES OF THE UNIVERSITY OF ILLINOIS, URBANA, IL (US)

(21) Appl. No.: 18/566,455

(22) PCT Filed: Jun. 6, 2022
(86) PCT No.: PCT/US22/32355

§ 371 Date: Dec. 1, 2023

Related U.S. Application Data
(60) Provisional application No. 63/292,021, filed on Dec. 21, 2021.
(60) Provisional application No. 63/197,168, filed on Jun. 4, 2021.

(51) Int. Cl. B01L 3/00; C12Q 1/70

(52) U.S. Cl. CPC B01L 3/502715; C12Q 1/70; B01L 2200/16; B01L 2300/0803; B01L 2300/0864; B01L 2300/126; B01L 2300/161; B01L 2400/02; B01L 2400/043

(57) ABSTRACT

The disclosure provides example devices and methods for virus and bacterium detection and identification. The example device includes (a) a housing having an air inlet and an air outlet, (b) a substrate disposed within the housing, where the at least one substrate has a through-slot, where the substrate has a plurality of wells, (c) an air-handling and precipitation chamber, a reagent and reaction chamber, and a detection chamber arranged in series and each coupled to the top surface of the substrate, (d) a first actuator coupled to the substrate and configured to rotate the substrate, (e) a second actuator coupled to and configured to rotate the air-handling and precipitation chamber, the reagent and reaction chamber, and the detection chamber, and (f) at least one processor electrically coupled to the air-handling and precipitation chamber, the reagent and reaction chamber, the detection chamber, the first actuator, and the second actuator.

### CROSS-REFERENCE TO RELATED APPLICATION

This international PCT application claims the benefit of the filing date of U.S. Provisional Patent Application No. 63/197,168, filed on Jun. 4, 2021, and U.S. Provisional Patent Application No. 63/292,021, filed on Dec. 21, 2021, which are hereby incorporated by reference in their entirety.

### BACKGROUND

COVID-19 has emerged as a major global health threat that has evolved into a global pandemic and the lack of treatment options at the outset underscores the importance of mitigation and the lack of current understanding of virus transmission through surveillance of airborne virus.

### SUMMARY

The disclosure provides a microfluidic device and methods of use to continuously sample and detect the presence of airborne viruses and bacteria, including mutant and wild-type SARS-Co-V-2, SARS-CoV, MERS, unknown CoV, known and unknown influenza strains, Respiratory Syncytial Virus, Mycobacterium tuberculosis, Bacillus anthracis or airborne pathogens circulating in animals. The device includes a detection system that samples the ambient air and facilitates a near-real time search for SARS-CoV-2 viral RNA or pathogen DNA for bacteria in the collected samples. In various implementations, a processor of the device may advantageously determine if a wildtype virus or a bacterium is present in the sampled air, determine the concentration, and may estimate distance to the viral or bacterial source based on the system's airflow, and perform analysis to compare the collected virus or bacteria with the known mutations. The system may beneficially perform screening of the virus to detect potentially unknown mutations in the spike protein. And amplified DNA samples may be beneficially kept for further sequencing later by a laboratory.

In brief, the microfluidic device and methods disclosed herein enable detection and mitigation of the spreading of airborne pathogens, such as SARS-CoV-2. With the rapid rise of mutations, the device and methods may be advantageously incorporated in local or global networks of bio-monitoring devices to prevent outbreak of further pandemics.

In a first aspect, an example microfluidic device is disclosed. The microfluidic device includes (a) a housing having an air inlet and an air outlet, (b) at least one substrate disposed within the housing in the form of a circular disk, where the at least one substrate has a through-slot extending radially between a central axis and an outer edge of the at least one substrate, where the at least one substrate has a plurality of wells arranged on a top surface of the at least one substrate, (c) an air-handling and precipitation chamber, a reagent and reaction chamber, and a detection chamber arranged in series and extending radially between the central axis and the outer edge of the at least one substrate, where the air-handling and precipitation chamber, the reagent and reaction chamber, and the detection chamber are each coupled to the top surface of the at least one substrate, (d) a first actuator coupled to the at least one substrate and configured to rotate the at least one substrate within the housing, (e) a second actuator coupled to and configured to rotate the air-handling and precipitation chamber, the reagent and reaction chamber, and the detection chamber relative to the top surface of the at least one substrate, and (f) at least one processor electrically coupled to the air-handling and precipitation chamber, the reagent and reaction chamber, the detection chamber, the first actuator, and the second actuator.

In a second aspect, an example method for using the microfluidic device according to the first aspect is disclosed. The microfluidic device used in the method according to the second aspect further utilizes a the plurality of wells that includes a first well, a second well, a third well, and a fourth well, where the at least one substrate has a circular microchannel and the first well, the second well, the third well, and the fourth well are arranged in series and connected by the circular microchannel on the top surface of the at least one substrate, and the microfluidic device further includes an electromagnet coupled to the at least one substrate and electrically coupled to the at least one processor.

The method of the second aspect includes (a) collecting a virus or a bacterium that is airborne, via the air-handling and precipitation chamber, and depositing at least one aqueous droplet or a crushed paper filter into the first well that contains a lysis buffer and magnetic beads coated with silica, (b) transferring magnetic beads coupled to vRNA or pathogen DNA, via the electromagnet and rotation of the at least one substrate, from the first well to the second well that contains a first washing buffer, (c) transferring the magnetic beads coupled to the vRNA or the pathogen DNA, via the electromagnet and rotation of the at least one substrate, from the second well to the third well that contains a second washing buffer, (d) transferring the magnetic beads coupled to the vRNA or the pathogen DNA, via the electromagnet and rotation of the at least one substrate, from the third well to the fourth well that contains an elution buffer, (e) removing the magnetic beads to a first exhaust via magnetic actuation and retaining the vRNA or the pathogen DNA in the fourth well via elution, (f) adding DNA amplification buffer in the fourth well, and (g) determining, via the processor, whether the virus or the bacterium is present in the fourth well based on either fluorescence of SYBER Green dye or affinity probes.

In a third aspect, an example method for using the microfluidic device according to the first aspect is disclosed. The method includes (a) collecting a virus or a bacterium that is airborne into an aqueous droplet at room temperature in a first well of the plurality of wells that contains a collection buffer, where the collection buffer comprises TBE: 100 mM Tris-HCl/pH 8.0, 90 mM boric acid, and 1 mM EDTA, (b) heating contents in the first well to 95° C. for 0-30 minutes and thereby lysing the virus or the bacterium, (c) adding, via electrokinetic or electroosomotic pumping, a DNA amplification buffer to the first well, where the amplification buffer comprises 1 U MMLV RT, 8 U Bst DNA Pol, 40 μM forward and reverse primers, in 1X Thermopol Buffer (New England Biolabs)+0.8M Betaine+1 μM SYBER Green, (d) heating the contents in the first well to 60-65° C. for 20-60 minutes, and (e) determining, via the processor, whether the virus or the bacterium is present in the first well based on either fluorescence of SYBER Green dye or affinity probes.

The features, functions, and advantages that have been discussed can be achieved independently in various examples or may be combined in yet other examples further details of which can be seen with reference to the following description and drawings.

### BRIEF DESCRIPTION OF THE DRAWINGS

FIG. 1 is a functional block diagram of a system using the microfluidic device, according to one example implementation;

FIG. 2 depicts a block diagram of a computing device and a computer network, according to an example implementation:

FIG. 3 is a perspective view of the microfluidic device, according to one example implementation:

FIG. 4 is a perspective view of a plurality of substrates arranged in a stack and the air-handling and precipitation chamber, a reagent and reaction chamber, and a detection chamber, according to one example implementation;

FIG. 5 shows is a perspective view of a substrate having a circular microchannel and a plurality of wells arrange in series, according to one example implementation:

FIG. 6 shows a mutant detection circuit, according to one example implementation:

FIG. 7A shows a top view of the mechanical iris and paper filter in the open position, according to an example implementation:

FIG. 7B shows a top view of the mechanical iris and paper filter in the closed position, according to an example implementation;

FIG. 8 shows a flowchart of a method for using the microfluidic device, according to an example implementation; and

FIG. 9 shows a flowchart of a method for using the microfluidic device, according to an example implementation.

The drawings are for the purpose of illustrating examples, but it is understood that the inventions are not limited to the arrangements and instrumentalities shown in the drawings

### DETAILED DESCRIPTION

### I. Overview

The disclosed examples provide a microfluidic device and methods of use to continuously sample and detect the presence of airborne viruses and bacteria, including mutant and wild-type SARS-Co-V-2, as well as methods to use the microfluidic device for both single-well and multi-well processing. The microfluidic device may be installed at transportation hubs and on airplanes, trains, cruise ships, and busses, for example. Additional sites could include other public and private spaces (e.g. hospitals, auditoriums, stadiums, hotels/dormitories, restaurants/bars, and office spaces etc.). In other embodiments, the devices may be worn by individuals. The microfluidic device and methods may advantageously reduce transmission of viruses and bacteria by providing real-time feedback to individuals, businesses, and other stakeholders. The real-time monitoring may also provided confidence in the safety of a given environment, thereby reducing the socio-economic ramifications of any virus and bacteria.

### II. Example Architecture

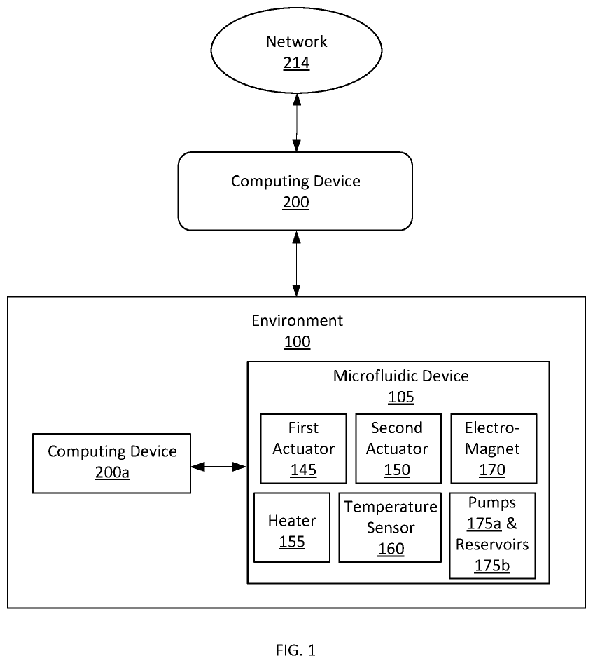

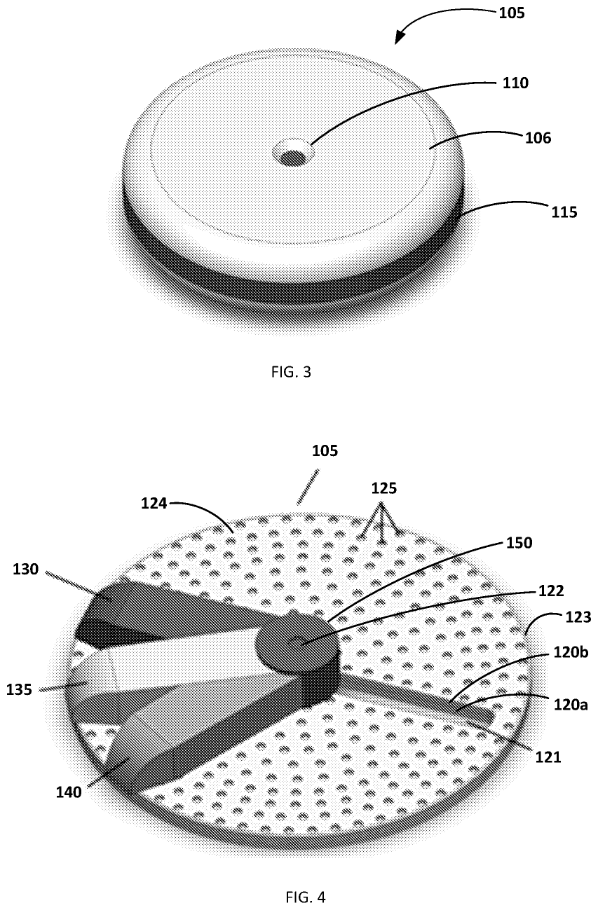

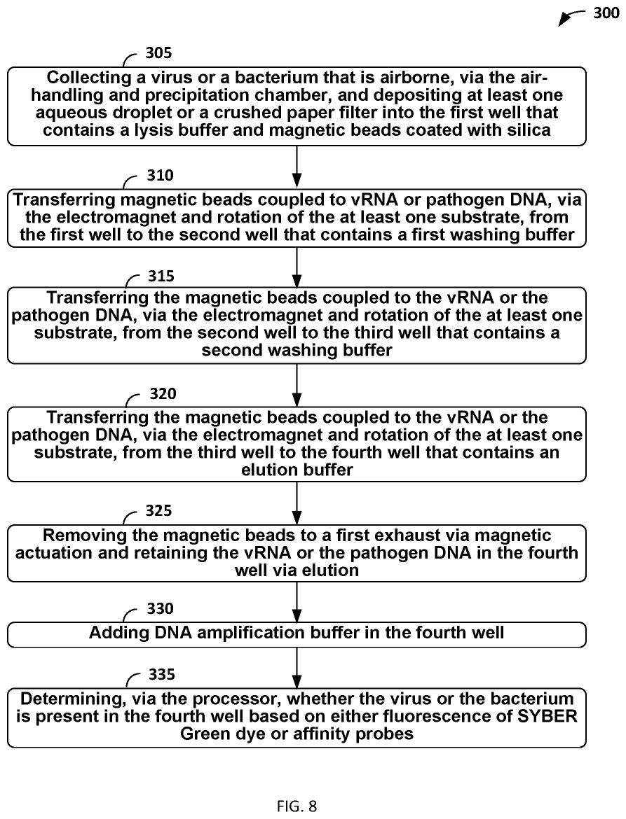

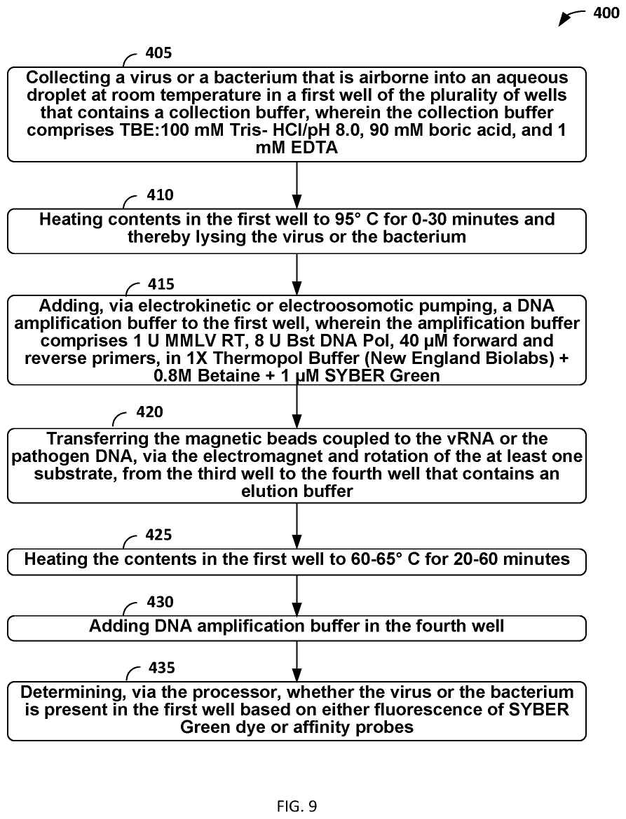

FIG. 1 is a block diagram showing an operating environment 100 that includes or involves, for example, a microfluidic device 105 shown in detail in FIGS. 3-7B and described below. Methods 300 and 400 in FIGS. 8-9 described below show embodiments of methods that can be implemented within this operating environment 100.

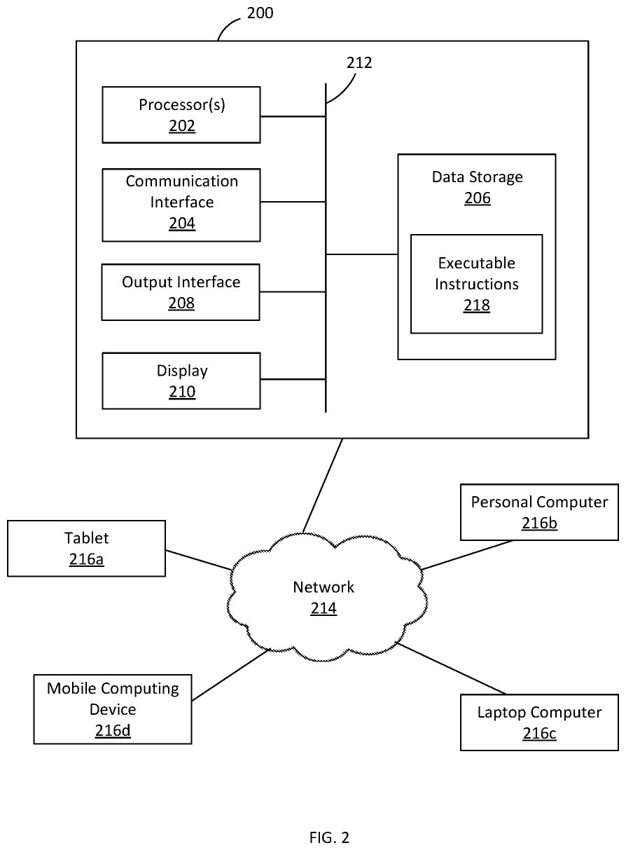

FIG. 2 is a block diagram illustrating an example of a computing device 200, according to an example implementation, that is configured to interface with operating environment 100, either directly or indirectly. The computing device 200 may be used to perform functions of the methods shown in FIGS. 8-9 and described below. In particular, computing device 200 can be configured to perform one or more functions, including determining whether a virus or a bacterium is present in an airborne sample and whether a mutant is present in the airborne sample, for example. The computing device 200 has a processor(s) 202, and also a communication interface 204, data storage 206, an output interface 208, and a display 210 each connected to a communication bus 212. The computing device 200 may also include hardware to enable communication within the computing device 200 and between the computing device 200 and other devices (e.g., not shown). The hardware may include transmitters, receivers, and antennas, for example.

The communication interface 204 may be a wireless interface and/or one or more wired interfaces that allow for both short-range communication and long-range communication to one or more networks 214 or to one or more remote computing devices 216 (e.g., a tablet 216a, a personal computer 216b, a laptop computer 216c and a mobile computing device 216d, for example). Such wireless interfaces may provide for communication under one or more wireless communication protocols, such as Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wired interfaces may include Ethernet interface, a Universal Serial Bus (USB) interface, or similar interface to communicate via a wire, a twisted pair of wires, a coaxial cable, an optical link, a fiber-optic link, or other physical connection to a wired network. Thus, the communication interface 204 may be configured to receive input data from one or more devices and may also be configured to send output data to other devices.

The communication interface 204 may also include a user-input device, such as a keyboard, a keypad, a touch screen, a touch pad, a computer mouse, a track ball and/or other similar devices, for example.

The data storage 206 may include or take the form of one or more computer-readable storage media that can be read or accessed by the processor(s) 202. The computer-readable storage media can include volatile and/or non-volatile storage components, such as optical, magnetic, organic or other memory or disc storage, which can be integrated in whole or in part with the processor(s) 202. The data storage 206 is considered non-transitory computer readable media. In some examples, the data storage 206 can be implemented using a single physical device (e.g., one optical, magnetic, organic or other memory or disc storage unit), while in other examples, the data storage 206 can be implemented using two or more physical devices.

The data storage 206 thus is a non-transitory computer readable storage medium, and executable instructions 218 are stored thereon. The instructions 218 include computer executable code. When the instructions 218 are executed by the processor(s) 202, the processor(s) 202 are caused to perform functions.

The processor(s) 202 may be a general-purpose processor or a special purpose processor (e.g., digital signal processors, application specific integrated circuits, etc.). The processor(s) 202 may receive inputs from the communication interface 204 and process the inputs to generate outputs that are stored in the data storage 206 and output to the display 210. The processor(s) 202 can be configured to execute the executable instructions 218 (e.g., computer-readable program instructions) that are stored in the data storage 206 and are executable to provide the functionality of the computing device 200 described herein.

The output interface 208 outputs information to the display 210 or to other components as well. Thus, the output interface 208 may be similar to the communication interface 204 and can be a wireless interface (e.g., transmitter) or a wired interface as well. The output interface 208 may send commands to one or more controllable devices, for example.

The computing device 200 shown in FIG. 2 may also be representative of a local computing device 200a in operating environment 100, for example, in communication with the microfluidic device 105. This local computing device 200a may perform one or more of the steps of the method 300 described below, may receive input from a user and/or may send image data and user input to computing device 200 to perform all or some of the steps of method 300.

FIGS. 8 and 9 show flowcharts of example methods 300 and 400 to determine whether a virus or a bacterium is present in an airborne sample, according to an example implementation. Methods 300 and 400 are example methods that could be used with the computing device 200 of FIG. 2, for example. In some instances, components of the devices and/or systems may be configured to perform the functions such that the components are configured and structured with hardware and/or software to enable such performance. Components of the devices and/or systems may be arranged to be adapted to, capable of, or suited for performing the functions, such as when operated in a specific manner. Methods 300 and 400 may include one or more operations, functions, or actions as illustrated by one or more of blocks 305-335 and blocks 405-435, respectively. Although the blocks are illustrated in a sequential order, some of these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of the present examples. In this regard, each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time such as register memory, processor cache and Random Access Memory (RAM). The computer readable medium may also include non-transitory media, such as secondary or persistent long-term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block in FIGS. 8 and 9, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

### III. Example Microfluidic Device

In a first aspect, shown in FIGS. 3-7B, a microfluidic device 105 includes a housing 106 having an air inlet 110 and an air outlet or exhaust 115. In one implementation, the housing 106 may have a diameter of about 30 cm. The device 105 also includes at least one substrate 120 disposed within the housing 106 in the form of a circular disk. In example implementations, the substrate may be formed from glass, silicon, or other known materials. The at least one substrate 120 has a through-slot 121 extending radially between a central axis 122 and an outer edge 123 of the at least one substrate 120. The at least one substrate 120 has a plurality of wells 125 arranged on a top surface 124 of the at least one substrate 120. In various optional implementations, surfaces of the plurality of wells 125 are coated with a material, such as Teflon, or patterned to induce selective hydrophobicity. And the top surface 124 of the at least one substrate 120 may also have a geometry designed to control thermal flux around the plurality of wells 125.

The device 100 further includes an air-handling and precipitation chamber 130, a reagent and reaction chamber 135, and a detection chamber 140 arranged in series and extending radially between the central axis 122 and the outer edge 123 of the at least one substrate 120. The air-handling and precipitation chamber 130, the reagent and reaction chamber 135, and the detection chamber 140 are each coupled to the top surface 124 of the at least one substrate 120. In one optional implementation, the air-handling and precipitation chamber 130 is configured for droplet deposition conducted via at least one of impaction, electrostatics, thermophoretics, photophoretics, and filtration.

In one optional implementation, with respect to impaction deposition into a liquid droplet, the air for the aerosol sample that potentially contains viral or bacterial matter is accelerated in an acceleration jet of an impactor include in the air-handling and precipitation chamber 130. The deposition of the virus- or bacteria-laden aerosol occurs in a droplet immobilized in a minor channel of the impactor that acts as the impaction point. A plurality of collection sites can be arranged in a grid or other parallel pattern. Impaction could be further enhanced be enhanced to deposit small, ultrafine virus- or bacteria-loaded aerosols by using electrophoretic deposition, by pre-charging the particles using a corona-based or other pre-charger.

Further, a condensation growth chamber could be arranged in the air-handling and precipitation chamber 130 before the acceleration jet of impactor to enhance or enable the deposition of small viral or bacterial particles. The condensation growth chamber contains a hot wet zone, where moisture will be added to the air-stream to increase humidity, and a growth region (supersaturated zone), where the moisture level exceeds 100% and starts condensing the aerosols in the condensation growth chamber. The enlarged aerosols will then be deposited onto the deposition site (i.e., a well 125) via impaction. Multiple condensation growth chambers can be fabricated/microfabricated in parallel to ensure sufficient flow rates.

Still further, in an optional implementation, thermophoretic deposition may be used to deposit the small viral or bacterial particles. For thermophoretically-enhanced impaction deposition the aerosol laden stream enters the air-handling and precipitation chamber 130 via the acceleration jet inlet of the impactor. The small aerosols are too small to deposit on the droplet solely via impaction, however the thermophoretic heater, that conformally envelopes the droplet, causes impaction of the viral-laden aerosol particle in the droplet.

In an alternative implementation, shown in FIGS. 7A-7B, the air-handling and precipitation chamber 130 is configured for droplet deposition conducted via filtration. Here, the air-handling and precipitation chamber 130 includes a mechanical iris configured to move between an open position and a closed position. In FIG. 7A, the mechanical iris 180 is configured such that a paper filter 185 is disposed in an opening of the mechanical iris 180 in the open position and configured to collect aerosols. The paper filter is configured to be crushed when the mechanical iris moves to the closed position, in FIG. 7B, such that the crushed paper filter is sized to be received in one of the plurality of wells. In operation, the mechanical iris moves between the open and closed positions through rotation of an outer ring.

In various implementations, the paper filter may include PC, PTFE, PES and PA. 2. In one optional implementation, the paper filter may be electrostatically pre-charged, via an electret, similar to N95 masks. For example, an electrostatic pre-charger could be used upstream of the airflow to increase additional deposition on an electret-based filter, thereby increasing the collection efficiency. Collection and detection of VLP surrogates of airborne virus with detection by microscopy (fluorescence) or RT-LAMP (colorimetric and fluorescence) has been demonstrated by the device 105 and methods 300 and 400 disclosed herein.

In an optional example implementation, as shown in FIG. 4, the at least one substrate 120 includes a plurality of substrates 120 arranged in a stack such that the air-handling and precipitation chamber 130, the reagent and reaction chamber 135, and the detection chamber 140 are configured to be rotated over the through-slots 121 of the plurality of substrates 120 to access substrates beneath 120a a top-most substrate 120b in the stack.

In addition, the device 100 includes a first actuator 145 coupled to the at least one substrate 120 and configured to rotate the at least one substrate 120 within the housing 106. The device 100 further includes a second actuator 150 coupled to and configured to rotate the air-handling and precipitation chamber 130, the reagent and reaction chamber 135, and the detection chamber 140 relative to the top surface 124 of the at least one substrate 120.

And the device 100 includes a computing device 200a having at least one processor 202 electrically coupled to the air-handling and precipitation chamber 130, the reagent and reaction chamber 135, the detection chamber 140, the first actuator 145, and the second actuator 150. The processor is discussed in section II above. The processor 202 and other components of the computing device 200 may be arranged in the housing 106 above the top surface 124 of the at least one substrate 120. A high-voltage power supply coupled to the processor and other power-driven components of the microfluidic device 105 may be similarly arranged within the housing 106.

In another optional example implementation, the device 100 includes at least one heater 155 coupled to the at least one substrate 120 and at least one temperature sensor 160 coupled to the at least one substrate 120. The at least one heater 155 and the at least one temperature sensor 160 are electrically coupled to the at least one processor 202. The heaters and sensors may be integrated in the substrates 120 for localized temperature control for nucleic acid amplification (NAT). Applicable NAT methods can include RT-PCR, RT-LAMP, and RT-PSR, or other suitable methods.

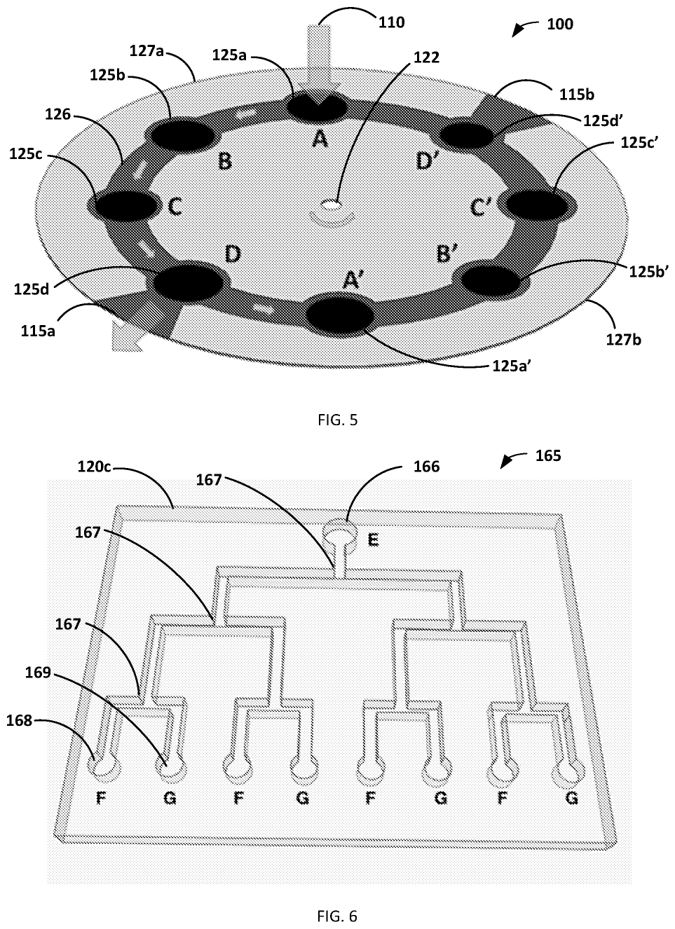

In one optional example implementation, as shown in FIG. 5, the plurality of wells 125 includes a first well 125a, a second well 125b, a third well 125c, and a fourth well 125d. The at least one substrate 120 has a circular microchannel 126 and the first well 125a, the second well 125b, the third well 125c, and the fourth well 125d are arranged in series and connected by the circular microchannel 126 on the top surface 124 of the at least one substrate 120. In a further optional example implementation, the plurality of wells 125 further includes a fifth well 125a′, a sixth well 125b′, a seventh well 125c′, and an eighth well 125d′ arranged in series and connected by the circular microchannel 126 on the top surface 124 of the at least one substrate 120. The first well 125a, the second well 125b, the third well 125c, and the fourth well 125d are arranged on a first half 127a of the at least one substrate 120 and the fifth well 125a′, the sixth well 125b′, the seventh well 125c′, and the eighth well 125d′ are arranged in opposing positions to the first well 125a, the second well 125b, the third well 125c, and the fourth well 125d, respectively, on a second half 127b of the at least one substrate 120. The foregoing arrangement permits multi-well analysis and detection of the virus or bacterium.

In an alternative implementation, shown in FIG. 3, the plurality of wells 125 may have a high-density arrangement such that there are 100 to 1000 wells per substrate 120. This high-density arrangement of wells may advantageously permit numerous single-well analysis and detection on each substrate 120 or numerous multi-well analysis and detection, where each row of wells are arranged in a series in circular bands extending between the central axis 122 and the outer edge 123 of the substrate 120 and are connected via a circular microchannel 126.

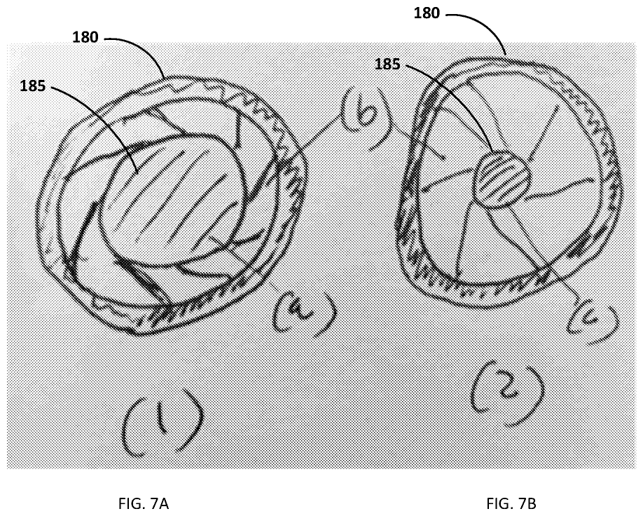

As shown in FIG. 6, the device 100 includes a mutant detection circuit 165. The mutant detection circuit 165 is arranged on a rotatable substrate arranged within the housing and disposed beneath the at least one substrate 120. While the rotatable substrate is shown as a polygon, in a preferred embodiment, the rotatable substrate 120c is circular. Further, the rotatable substrate 120c may have the same physical properties of the at least one substrate 120 discussed above and the mutant detection circuit 165 may be embedded or etched in the rotatable substrate 120c. In one implementation, the rotatable substrate 120c is fabricated in glass.

The mutant detection circuit 165 includes a deposition site 166 coupled to a plurality of droplet-dividing electrode gates 167 that are coupled to a plurality of amplification sites 168 and a plurality of detection sites 169. In the present example, there are three sets of electrode gates 167 arranged to divide an initial droplet into four pairs of amplification sites 168 and detection sites 169. In an alternative implementation, the mutant detection circuit may have up to 300 pairs of amplification and detections sites 168, 169 and additional buffer could be pumped into the system from a reservoir 175b via one of pumps 175a during droplet division. The 300-pair embodiment could be used to assay a 100 amino acid region of the spike RBD, a known hotspot for mutations that evade the immune system to specifically identify the mutant.

The plurality of amplification sites 168 contain mutant site primers and the plurality of detection sites 169 contain complementary wild-type primers. In operation, the mutant detection circuit 165 is configured to receive a solution comprising Reverse Transcriptase/Polymerase, primers, and a virus or bacterium sample from the eighth well 125d′ after the virus or the bacterium has been detected in the fourth well 125d. The example mutant detection circuit 165 in FIG. 6 permits detection of up to four mutants and contains eight parallel sites. The rotatable substrate 120c is configured to use centripetal forces to promote pumping of the sample through the mutant detection circuit. In a further implementation, heaters are coupled to or integrated within the rotatable substrate 120c around the amplification sites 168 and detection sites 169.

In various implementations of the mutant detection circuit, the initial amplification could be PCR or LAMP based. Moreover, the variant detection could be conducted by PCR or LAMP. In the case of PCR, forward or reverse primers are designed to be complementary to the wild-type or mutant sequence. In the case of LAMP, one of the 6 primers is designed to be complementary to the wild-type or the mutant sequence.

In a further optional implementation, the device 100 includes an electromagnet 170 coupled to the at least one substrate 120 and electrically coupled to the at least one processor 202. The electromagnet 170 is configured to interact with magnetic beads disposed in at least one of the plurality of wells 125 of the at least one substrate 120. In one implementation, the electromagnet 170 may be arranged beneath the at least one substrate 120 or substrate stack within the housing 106.

In one optional implementation, the device 100 includes a plurality of pumps 175a and reservoirs 175b disposed within the housing 106 and in fluid communication with at least one of the plurality of wells 125 of the at least one substrate 120. The plurality of pumps 175a are electrically coupled to the at least one processor 202. In various embodiments, the reservoirs 175b contain lysis buffers, washing buffers, elution buffers, primers, and solutions containing magnetic beads. The pumps 175a are conventional miniature pumps known in the art. In one implementation, the pumps 175a and reservoirs 175b may be contained within the reagent and reaction chamber 135. In alternative optional implementations, liquid handling utilizes pressure driven microfluidic pumping, electrohydrodynamic pumping techniques, or a combination of both applied to liquid valving and volume control, routing, waste handling and heat transfer that is required for lab-on-a-chip functionality. Liquid flow paths and channels on the at least one substrate 120 may be prepared via surface machining, soft lithography methods and/or additive or other suitable manufacturing techniques.

In a further optional implementation, the at least one substrate 120 includes paper having a reagent embedded in at least one of the plurality of wells 120. In this implementation, the microfluidic device 105 further includes at least one pressure-based actuator or electrostatic actuator configured to seal a wetted-area on the paper substrate.

The following methods 300 and 400 may include one or more operations, functions, or actions as illustrated by one or more of blocks 305-335 and 405-435. Although the blocks are illustrated in a sequential order, these blocks may also be performed in parallel, and/or in a different order than those described herein. Also, the various blocks may be combined into fewer blocks, divided into additional blocks, and/or removed based upon the desired implementation. Alternative implementations are included within the scope of the examples of the present disclosure in which functions may be executed out of order from that shown or discussed, including substantially concurrent or in reverse order, depending on the functionality involved, as would be understood by those reasonably skilled in the art.

Referring now to FIG. 3, FIG. 3 shows a flowchart of an example method 300 for using the microfluidic device 100, according to an example implementation. In this implementation, the plurality of wells 125 includes a first well 125a, a second well 125b, a third well 125c, and a fourth well 125d. The at least one substrate 120 has a circular microchannel 126, and the first well 125a, the second well 125b, the third well 125c, and the fourth well 125d are arranged in series and connected by the circular microchannel 126 on the top surface 124 of the at least one substrate 120. The device 100 also includes an electromagnet 170 coupled to the at least one substrate 120 and electrically coupled to the at least one processor 202.

Method 300 includes, at block 305, collecting a virus or a bacterium that is airborne, via the air-handling and precipitation chamber 130, and depositing at least one aqueous droplet or a crushed paper filter into the first well 125a that contains a lysis buffer and magnetic beads coated with silica. The lysis buffer and magnetic beads may be preloaded in the first well 125a or supplied from the plurality of reservoirs 175b via the plurality of pumps 175a based on a signal from the processor 202. In operation, the lysis buffer inactivates the virus or bacterium and thereby mitigates biosafety concerns for the collected virus or bacterium. Then, at block 310, the magnetic beads coupled to vRNA or pathogen DNA are transferred, via the electromagnet 170 and rotation of the at least one substrate 120, from the first well 125a to the second well 125b that contains a first washing buffer. The vRNA or pathogen DNA may be bonded to the magnetic beads through various known reactions. In operation, the at least one substrate 120 is rotated via the first actuator 145, and actuation of the electromagnet 170 holds the magnetic beads in place such that magnetic beads move through the microchannel 126 to the next well 125 in the series. In addition, the reagent and reaction chamber 135 is likewise rotated over the top of the second well 125b via the second actuator 150. The first washing buffer may be preloaded or supplied to the second well 125b from one of a plurality of reservoirs 175b via one of a plurality of pumps 175a.

Next, at block 315, the magnetic beads coupled to the vRNA or the pathogen DNA are transferred, via the electromagnet 170 and rotation of the at least one substrate 120, from the second well 125b to the third well 125c that contains a second washing buffer. Here, the reagent and reaction chamber 135 will be positioned over the top of the third well 125c due to partial rotation of the at least one substrate 120 to transfer the magnetic beads to the third well 125c. The second washing buffer may be preloaded or supplied to the third well 125c from another one of the plurality of reservoirs 175b via one of the plurality of pumps 175a.

And, at block 320, the magnetic beads coupled to the vRNA or the pathogen DNA are transferred, via the electromagnet 170 and rotation of the at least one substrate 120, from the third well 125c to the fourth well 125d that contains an elution buffer. In addition, the detection chamber 140 is likewise rotated over the fourth well 125d via the second actuator 150. The elution buffer may be preloaded or supplied to the fourth well 125d from another one of the plurality of reservoirs 175b via one of the plurality of pumps 175a. At block 325, the magnetic beads are removed to a first exhaust 115a via magnetic actuation and the vRNA or the pathogen DNA is retained in the fourth well 125d via elution.

Then, at block 330, DNA amplification buffer is added in the fourth well 125d. DNA amplification buffer is supplied from another one of the plurality of reservoirs 175b via one of the plurality of pumps 175a. And, at block 335, the processor 202 determines whether the virus or the bacterium is present in the fourth well 125d based on either fluorescence of SYBER Green dye or affinity probes. In various implementations, detection of the virus or the bacterium may be based on fluorescence, luminescence, colorimetric, or turbity properties. In the detection chamber 140, optical detection using an excitation light source may conducted using a photodetector and/or a miniature photomultiplier for increased sensitivity.

In addition, when primers are used that contain tags at their 5′, affinity probes (e.g., anti-FITC antibodies) may also be used for detection. PSR oligonucleotide primers are designed to contain ˜20 nt that are complementary to the sequence of interest and external botanical sequences at the 5′ end (acgaattcgtacatagaagtatag and gatatgaagatacatgcttaagca for the forward and reverse primers, respectively). The forward and reverse primers target sequences that are typically ˜200 nt apart. For the detection of wild-type SARS-CoV-2, primer sequences are designed from the RdRP, N, E, and S genes with consideration of splice variants, Tm, 3′ stability, secondary structure, primer-dimer formation and non-specific binding to other sites.

Following are two optional examples of primers that may be used to detect specific mutations in the SARS-CoV-2 spike gene. PCR or LAMP primers are designed to be complementary (5′ or forward primer) or non-complementary (3′ or reverse primer) to the wildtype or mutant sequence with the mismatch in the primer at the beginning, the end or in the middle. For example, the N501Y mutation is due to the codon changing from AAT=>TAT. As such, a wildtype primer could be cctttaca atcatatggt ttccaaccca cta and the mutant primer could be cctttaca atcatatggt ttccaaccca ctt. Alternatively, the complementary primer could be to the wildtype sequence aatggtgt tggttaccaa ccatacagag and the mutant primer could be tatggtgt tggttaccaa ccatacagag. In another example, the D614G mutation is due to the codon changing from GAT=>GGT. As such, a wildtype primer could be ctaac caggttgctg ttctttatca gga and the mutant primer could be ctaac caggttgctg ttctttatca ggg. Alternatively, the complementary primer could be to the wildtype sequence atgttaac tgcacagaag tccctgttg and the mutant primer gtgttaac tgcacagaag tccctgttg.

For detection of unknown mutations or deletions, primers may be designed complementary to all possible mutations at a given site. For example, in the case of N501 the primers could be be cctttaca atcatatggt ttccaaccca cta (wildtype), cctttaca atcatatggt ttccaaccca ctg (N501D unknown mutant), cctttaca atcatatggt ttccaaccca ctc (N501H unknown mutant), cctttaca atcatatggt ttccaaccca ctt (N501Y-known mutant). Additional mutations could be assessed with primers complementary or noncomplementary at the second and third sites of the codon. For example, in the case of N501 the primers targeting the second base of the codon could be cctttaca atcatatggt ttccaaccca ctaa (wildtype), cctttaca atcatatggt ttccaaccca ctag (N501S unknown mutant), cctttaca atcatatggt ttccaaccca ctac (N501S unknown mutant), cctttaca atcatatggt ttccaaccca ctat (N501M unknown mutant).

In one optional implementation, method 300 further includes mixing, via electrowetting, the at least one aqueous droplet with the lysis buffer and magnetic beads in the first well 125a at room temperature for at least 10 minutes. In this example, the lysis buffer includes 4% NH4SO4, 0.8% NP-40 in 0.2 M Tris Acetate/pH 4, and proteinase K at 1 mg/ml. Then, the magnetic beads coupled to the vRNA or the pathogen DNA are washed via electrowetting at room temperature for at least 10 minutes in the second well 125b. In this example, the first washing buffer includes 0.5% NP-40 in 0.01 M Tris-HCl pH 6.8 and proteinase K at 1 mg/ml. Next, the magnetic beads coupled to the vRNA or the pathogen DNA are washed via electrowetting at room temperature for at least 10 minutes in the third well 125c. In this example, the second washing buffer includes 0.5% NP-40 in 0.01 M Tris-HCl pH 6.8. Then, the magnetic beads coupled to the vRNA or the pathogen DNA are mixed via electrowetting at room temperature for at least 10 minutes in the elution buffer in the fourth well 125d. In this example, the elution buffer includes 10 mM Tris HCl pH 8.5. mixing, electrokinetics, the vRNA or the pathogen DNA and the DNA amplification buffer at a temperature ranging from 60-65° C. for 20-30 minutes. In this example, the DNA amplification buffer includes 1 U MMLV RT, 8 U Bst DNA Pol, 40 UM forward and reverse primers, in 1X Thermopol Buffer (New England Biolabs)+0.8M Betaine+1 μM SYBER Green. Lyophylized DNA amplification buffer, including enzymes, has been shown to be stable for 6 months or more at 4° C.

In one optional implementation, method 300 further includes extracting amplified viral or bacterial DNA solution from the fourth well 125d to one of a plurality of reservoirs 175b.

In one optional implementation, the plurality of wells 125 comprises a fifth well 125a′, a sixth well 125b′, a seventh well 125c′, and an eighth well 125d′ arranged in series and connected by the circular microchannel 126 on the top surface 124 of the at least one substrate 120. The first well 125a, the second well 125b, the third well 125c, and the fourth well 125d are arranged on a first half 127a of the at least one substrate 120 and the fifth well 125a′, the sixth well 125b′, the seventh well 125c′, and the eighth well 125d′ are arranged in opposing positions to the first well 125a, the second well 125b, the third well 125c, and the fourth well 125d, respectively, on a second half 127b of the at least one substrate 120.

With the device having the foregoing features, method 300 further includes collecting a virus or a bacterium that is airborne, via the air-handling and precipitation chamber 130, and depositing at least one aqueous droplet or a crushed paper filter into the fifth well 125a′ that contains the lysis buffer and magnetic beads coated with silica. The lysis buffer and magnetic beads may be preloaded or pumped from reservoirs 175b to the fifth well 125a′, in response to a signal from the processor 202. The magnetic beads coupled to vRNA or pathogen DNA are then transferred via the electromagnet 170 and rotation of the at least one substrate 120 from the fifth well 125a′ to the sixth well 125b′ that contains the first washing buffer. As noted above, the first washing buffer may be preloaded or pumped to the sixth well 125b′ from a reservoir 175b. Next, the magnetic beads coupled to the vRNA or the pathogen DNA are transferred via the electromagnet 170 and rotation of the at least one substrate 120 from the sixth well 125b′ to the seventh well 125c′ that contains the second washing buffer. Again, the second washing buffer may be preloaded or pumped to the seventh well 125c′ from a reservoir 175b. The magnetic beads coupled to the vRNA or the pathogen DNA are then transferred via the electromagnet and rotation of the at least one substrate from the seventh well 125c′ to the eighth well 125d′ that contains the elution buffer. The elution buffer may be preloaded or pumped from a reservoir 175b to the eighth well 125d′. Finally, the magnetic beads are removed to a second exhaust 115b via magnetic actuation and the vRNA or the pathogen DNA is retained in the eighth well 125d′ via elution.

Utilizing the complementary wells 125a-d′ in the foregoing steps allows virus or bacteria collection to be performed in the fifth well 125a′ in parallel with DNA amplification and analysis in fourth well 125d to increase reliability of detected results and to reduce overall detection time. Alternatively, complementary wells 125a′-d′ may allow staggered collection times. For example, an airborne sample may be collected in the first well 125a at a first point in time for ten minutes, and an airborne sample may be collected in the fifth well 125a′ at a second point in time (e.g., five minutes after the first time period) for ten minutes.

In one optional implementation, when the collection, mixing, washing, amplification, and detection steps are performed in wells 125a-d, similar steps may be performed in parallel with wells 125a′-d′. In this implementation, the processor 202 determines whether the virus or the bacterium is present in the fourth well 125d. If the processor 202 determines that the virus or the bacterium is present in the fourth well 125, then a solution including Reverse Transcriptase/Polymerase and primers is added to the vRNA or the pathogen DNA in the eighth well 125d′. The first actuator 145 rotates a mutant detection circuit 165 on a rotatable substrate 120c arranged within the housing 106 and disposed beneath the at least one substrate 120a-b such that a deposition site 166 of the mutant detection circuit 165 is arranged under the through-hole 121 of the at least one substrate 120a-b. The solution in the eighth well 125d′, including the vRNA or the pathogen DNA, is transferred to the deposition site 166 of the mutant detection circuit 165, via electrodynamic pumping. The rotatable substrate 120c is rotated to induce centripetal force in the mutant detection circuit 165 such that the transferred solution is pumped from the deposition site 166 through a plurality of droplet-dividing electrode gates 167 until the transferred solution advances to a plurality of amplification sites 168 and a plurality of detection sites 169.

The plurality of amplification sites 168 contain mutant site primers and the plurality of detection sites contain complementary wild-type primers, as discussed above. Upon wetting of the mutant site primers and the wild-type primers, the plurality of amplification sites 168 and the plurality of detection sites 169 are heated to a temperature ranging from 60° C. to 65° C. Alternatively, thermal cycling for PCR could be utilized such that the amplification and detection sites are heated to 95° C., 55° C., and 65° C. Signals for turbidity and conductivity are detected, via a charge-coupled device (“CCD”) that is included in the detection chamber 140 to image the combination of signals at a given pair of amplification and detection sites 168, 169. The processor 202 determines based on the detected signals, whether a mutant is present in at least one of the plurality of detection sites 169 and identifies a type of the mutant in at least one of the plurality of amplification sites 168.

Multiple pathogens may be detected simultaneously using pathogen-specific primers. The lysis and handling buffers will be similar but may be optimized. For example, in some cases (SARS), detergent is not needed, and heating the sample in a LAMP buffer will suffice. In the case of other pathogens, more extreme conditions with detergent and a preheating cycle may be needed to disrupt the viral or bacterial membrane.

Alternatively, if the processor 202 determines that the virus or the bacterium is not present in the fourth well 125d, then DNA amplification buffer is added in the eighth well 125d′. The processor 202 determining whether a virus or a bacterium is present in the eighth well 125d′ based on either fluorescence of SYBER Green dye or affinity probes. The detection occurs in the detection chamber 140 arranged over the eighth well 125d″.

In an alternative implementation, the collection, transferring, mixing, washing steps are performed in wells 125a′-d′ during a time period ranging from 5 to 30 minutes after an initial collection time associated with collecting the virus or bacterium that is airborne, via the air-handling and precipitation chamber 130. Then, DNA amplification buffer is added in the eighth well 125d′. The processor 202 then determines whether a virus or a bacterium is present in the eighth well 125d′ based on either fluorescence of SYBER Green dye or affinity probes. In various implementations, airborne collection could take place from 10 minutes to hours and the assay may take place for 20-60 minutes. Again, collection times could be staggered (e.g., first well 125a collects at time 0-60 minutes, fifth well 125a′ collects at 10-70 minutes, an additional well collects at time 20-80 etc. to provide real-time detection in10 minute intervals).

In another optional implementation, the second actuator rotates the reagent and reaction chamber 135 over the second well 125b after transferring the magnetic beads coupled to the vRNA or the pathogen DNA to the second well 125b. Then the second actuator rotates the reagent and reaction chamber 135 over the third well 125c after transferring the magnetic beads coupled to the vRNA or the pathogen DNA to the third well 125c. In an alternative embodiment, the reagent and reaction chamber 135 remains in place and the third well 125c is advanced underneath via rotation of the at least one substrate 120. Next, the second actuator 150 rotates the detection chamber 145 over the fourth well 125d after transferring the magnetic beads coupled to the vRNA or the pathogen DNA to the fourth well 125d.

Referring now to FIG. 4, FIG. 4 shows a flowchart of an example method 400 for using the device 100, according to an example implementation. At block 405, method 400 includes collecting a virus or a bacterium that is airborne into an aqueous droplet at room temperature in a first well 125a of the plurality of wells 125 that contains a collection buffer. The collection buffer includes TBE: 100 mM Tris-HCl/pH 8.0, 90 mM boric acid, and 1 mM EDTA. Contents in the first well are heated to 95° C. for 0-30 minutes, thereby lysing the virus or the bacterium. At block 410, electrokinetic or electroosomotic pumping is used to add a DNA amplification buffer to the first well 125a. The amplification buffer includes 1 U MMLV RT, 8 U Bst DNA Pol, 40 μM forward and reverse primers, in 1X Thermopol Buffer (New England Biolabs)+0.8M Betaine+1 μM SYBER Green. Then, at block 415, the contents in the first well 125a are heated to 60-65° C. for 20-60 minutes. Next, at block 420, the processor 202 determines whether the virus or the bacterium is present in the first well 125a based on either fluorescence of SYBER Green dye or affinity probes.

In one optional implementation, microfluidic device includes a second well 125b that contains a solution including viral gene DNA and a third well 125c that contains a solution containing buffer. In this implementation, the second well 125b acts as a positive control and the third well 125c acts as negative control. Method 400 further includes heating the solution in the second well 125b and the solution in the third well 125c to 95° C. for 0-30 minutes. Electrokinetic or electroosomotic pumping is used to add a DNA amplification buffer to the second well 125b and to the third well 125c. The contents in the second well 125b and the third well 125c are then heated to 60-65° C. for 20-60 minutes. And the processor 202 determines whether the virus or the bacterium is present in the second well 125a and the third well 125c based on either fluorescence of SYBER Green dye or affinity probes.

In one optional implementation, method 400 includes extracting amplified viral DNA solution from the first well 125a to a reservoir 175b.

In an optional implementation, methods 300 and 400 may include sending a signal, via the processor 202, to a local detection device indicating detection of the virus or the bacterium and thereby causing the local detection device to emit an alert in the form of at least one of a light or a sound.

In another optional implementation, methods 300 and 400 may include receiving, via the processor 202, at least one signal from a smartphone 216d or computing device 216c associated with an airborne viral sample to enable alerts for a detected virus or bacterium and geotracking.

In yet another optional implementation, methods 300 and 400 may include sending a signal, via the processor 202, containing virus detection information to a remote database.

1. A microfluidic device, comprising:
a housing having an air inlet and an air outlet;
at least one substrate disposed within the housing in the form of a circular disk, wherein the at least one substrate has a through-slot extending radially between a central axis and an outer edge of the at least one substrate, wherein the at least one substrate has a plurality of wells arranged on a top surface of the at least one substrate;
an air-handling and precipitation chamber, a reagent and reaction chamber, and a detection chamber arranged in series and extending radially between the central axis and the outer edge of the at least one substrate, wherein the air-handling and precipitation chamber, the reagent and reaction chamber, and the detection chamber are each coupled to the top surface of the at least one substrate;
a first actuator coupled to the at least one substrate and configured to rotate the at least one substrate within the housing;
a second actuator coupled to and configured to rotate the air-handling and precipitation chamber, the reagent and reaction chamber, and the detection chamber relative to the top surface of the at least one substrate; and
at least one processor electrically coupled to the air-handling and precipitation chamber, the reagent and reaction chamber, the detection chamber, the first actuator, and the second actuator.

2. The microfluidic device of claim 1, wherein the at least one substrate comprises a plurality of substrates arranged in a stack such that the air-handling and precipitation chamber, the reagent and reaction chamber, and the detection chamber are configured to be rotated over the through-slots of the plurality of substrates to access substrates beneath a top-most substrate in the stack.

3. The microfluidic device of claim 1, further comprising
at least one heater coupled to the at least one substrate; and
at least one temperature sensor coupled to the at least one substrate, wherein the at least one heater and the at least one temperature sensor are electrically coupled to the at least one processor.

4. The microfluidic device of claim 1, wherein the plurality of wells comprises a first well, a second well, a third well, and a fourth well, wherein the at least one substrate has a circular microchannel and the first well, the second well, the third well, and the fourth well are arranged in series and connected by the circular microchannel on the top surface of the at least one substrate.

5. The microfluidic device of claim 4, wherein the plurality of wells comprises a fifth well, a sixth well, a seventh well, and an eighth well arranged in series and connected by the circular microchannel on the top surface of the at least one substrate, wherein the first well, the second well, the third well, and the fourth well are arranged on a first half of the at least one substrate and the fifth well, the sixth well, the seventh well, and the eighth well are arranged in opposing positions to the first well, the second well, the third well, and the fourth well, respectively, on a second half of the at least one substrate.

6. The microfluidic device of claim 5, further comprising:
a mutant detection circuit on a rotatable substrate arranged within the housing and disposed beneath the at least one substrate, wherein the mutant detection circuit comprises a deposition site coupled to a plurality of droplet-dividing electrode gates that are coupled to a plurality of amplification sites and a plurality of detection sites, wherein the plurality of amplification sites contain mutant site primers and the plurality of detection sites contain complementary wild-type primers, wherein the mutant detection circuit is configured to receive a solution comprising Reverse Transcriptase/Polymerase, primers, and a virus or bacterium sample from the eighth well after the virus or the bacterium has been detected in the fourth well.

7. The microfluidic device of claim 1, further comprising:
an electromagnet coupled to the at least one substrate and electrically coupled to the at least one processor, wherein the electromagnet is configured to interact with magnetic beads disposed in at least one of the plurality of wells of the at least one substrate.

8. The microfluidic device of claim 1, further comprising:
a plurality of pumps and reservoirs disposed within the housing and in fluid communication with at least one of the plurality of wells of the at least one substrate, wherein the plurality of pumps are electrically coupled to the at least one processor.

9. The microfluidic device of claim 1, wherein surfaces of the plurality of wells are coated with a material or patterned to induce selective hydrophobicity.

10. The microfluidic device of claim 1, wherein the top surface of the at least one substrate has a geometry designed to control thermal flux around the plurality of wells.

11. The microfluidic device of claim 1, wherein the at least one substrate comprises paper having a reagent embedded in at least one of the plurality of wells, the microfluidic device further comprising at least one pressure-based actuator or electrostatic actuator configured to seal a wetted-area on the paper substrate.

12. The microfluidic device of claim 1, wherein the air-handling and precipitation chamber is configured for droplet deposition conducted via at least one of impaction, electrostatics, thermophoretics, photophoretics, and filtration.

13. The microfluidic device of claim 12, wherein the air-handling and precipitation chamber is configured for droplet deposition conducted via filtration, wherein the air-handling and precipitation chamber comprises a mechanical iris configured to move between an open position and a closed position, wherein the mechanical iris is configured such that a paper filter is disposed in an opening of the mechanical iris in the open position, and wherein the paper filter is configured to be crushed when the mechanical iris moves to the closed position such that the crushed paper filter is sized to be received in one of the plurality of wells.

14. A method for using the microfluidic device of claim 1, wherein the plurality of wells comprises a first well, a second well, a third well, and a fourth well, wherein the at least one substrate has a circular microchannel and the first well, the second well, the third well, and the fourth well are arranged in series and connected by the circular microchannel on the top surface of the at least one substrate, wherein the microfluidic device further comprises an electromagnet coupled to the at least one substrate and electrically coupled to the at least one processor, the method comprising:
collecting a virus or a bacterium that is airborne, via the air-handling and precipitation chamber, and depositing at least one aqueous droplet or a crushed paper filter into the first well that contains a lysis buffer and magnetic beads coated with silica;
transferring magnetic beads coupled to vRNA or pathogen DNA, via the electromagnet and rotation of the at least one substrate, from the first well to the second well that contains a first washing buffer;
transferring the magnetic beads coupled to the vRNA or the pathogen DNA, via the electromagnet and rotation of the at least one substrate, from the second well to the third well that contains a second washing buffer;
transferring the magnetic beads coupled to the vRNA or the pathogen DNA, via the electromagnet and rotation of the at least one substrate, from the third well to the fourth well that contains an elution buffer;
removing the magnetic beads to a first exhaust via magnetic actuation and retaining the vRNA or the pathogen DNA in the fourth well via elution;
adding DNA amplification buffer in the fourth well; and
determining, via the processor, whether the virus or the bacterium is present in the fourth well based on either fluorescence of SYBER Green dye or affinity probes.

15. The method of claim 14, further comprising:
mixing, via electrowetting, the at least one aqueous droplet with the lysis buffer and magnetic beads in the first well at room temperature for at least 10 minutes, wherein the lysis buffer comprises 4% NH4SO4, 0.8% NP-40 in 0.2 M Tris Acetate/pH 4, and proteinase K at 1 mg/ml;
washing, via electrowetting, the magnetic beads coupled to the vRNA or the pathogen DNA at room temperature for at least 10 minutes in the second well, wherein the first washing buffer comprises 0.5% NP-40 in 0.01 M Tris-HCl pH 6.8 and proteinase K at 1 mg/ml;
washing, via electrowetting, the magnetic beads coupled to the vRNA or the pathogen DNA at room temperature for at least 10 minutes in the third well, wherein the second washing buffer comprises 0.5% NP-40 in 0.01 M Tris-HCl pH 6.8;
mixing, via electrowetting, the magnetic beads coupled to the vRNA or the pathogen DNA at room temperature for at least 10 minutes in the elution buffer in the fourth well, wherein the elution buffer comprises 10 mM Tris HCl pH 8.5;
mixing, via electrokinetics, the vRNA and the DNA amplification buffer at a temperature ranging from 60-65° C. for 20-30 minutes, wherein the DNA amplification buffer comprises 1 U MMLV RT, 8 U Bst DNA Pol, 40 μM forward and reverse primers, in 1× Thermopol Buffer (New England Biolabs)+0.8M Betaine+1 μM SYBER Green.

16. The method of claim 14, further comprising
extracting amplified viral or bacterial DNA solution from the fourth well to a reservoir.

17. The method of claim 14, wherein the plurality of wells comprises a fifth well, a sixth well, a seventh well, and an eighth well arranged in series and connected by the circular microchannel on the top surface of the at least one substrate, wherein the first well, the second well, the third well, and the fourth well are arranged on a first half of the at least one substrate and the fifth well, the sixth well, the seventh well, and the eighth well are arranged in opposing positions to the first well, the second well, the third well, and the fourth well, respectively, on a second half of the at least one substrate, the method further comprising:
collecting a virus or a bacterium that is airborne, via the air-handling and precipitation chamber, and depositing at least one aqueous droplet or a crushed paper filter into the fifth well that contains the lysis buffer and magnetic beads coated with silica;
transferring the magnetic beads coupled to vRNA or pathogen DNA, via the electromagnet and rotation of the at least one substrate, from the fifth well to the sixth well that contains the first washing buffer;
transferring the magnetic beads coupled to the vRNA or the pathogen DNA, via the electromagnet and rotation of the at least one substrate, from the sixth well to the seventh well that contains the second washing buffer;
transferring the magnetic beads coupled to the vRNA or the pathogen DNA, via the electromagnet and rotation of the at least one substrate, from the seventh well to the eighth well that contains the elution buffer; and
removing the magnetic beads to a second exhaust via magnetic actuation and retaining the vRNA or the pathogen DNA in the eighth well via elution.

18. The method of claim 17, the method further comprising:
performing the steps of claim 17 in parallel with the following steps (a)-(g);
(a) collecting the virus or the bacterium that is airborne, via the air-handling and precipitation chamber, and depositing the at least one aqueous droplet or the crushed paper filter into the first well that contains the lysis buffer and magnetic beads coated with silica;
(b) transferring the magnetic beads coupled to vRNA or pathogen DNA, via the electromagnet and rotation of the at least one substrate, from the first well to the second well that contains the first washing buffer;
(c) transferring the magnetic beads coupled to the vRNA or the pathogen DNA, via the electromagnet and rotation of the at least one substrate, from the second well to the third well that contains the second washing buffer;
(d) transferring the magnetic beads coupled to the vRNA or the pathogen DNA, via the electromagnet and rotation of the at least one substrate, from the third well to the fourth well that contains the elution buffer;
(e) removing the magnetic beads to the first exhaust via magnetic actuation and retaining the vRNA or the pathogen DNA in the fourth well via elution;
(f) adding the DNA amplification buffer in the fourth well; and
(g) determining, via the processor, whether the virus or the bacterium is present in the fourth well based on either the fluorescence of SYBER Green dye or the affinity probes:
determining, via the processor, whether the virus or the bacterium is present in the fourth well:
if the processor determines that the virus or the bacterium is present in the fourth well,
(i) adding a solution comprising Reverse Transcriptase/Polymerase and primers to the vRNA or the pathogen DNA in the eighth well;
(ii) rotating, via the first actuator, a mutant detection circuit on a rotatable substrate arranged within the housing and disposed beneath the at least one substrate such that a deposition site of the mutant detection circuit is arranged under the through-hole of the at least one substrate;
(iii) transferring the solution in the eighth well, including the vRNA or the pathogen DNA, to the deposition site of the mutant detection circuit, via electrodynamic pumping;
(iv) rotating the rotatable substrate to induce centripetal force in the mutant detection circuit such that the transferred solution is pumped from the deposition site through a plurality of droplet-dividing electrode gates until the transferred solution advances to a plurality of amplification sites and a plurality of detection sites, wherein the plurality of amplification sites contain mutant site primers and the plurality of detection sites contain complementary wild-type primers;
(v) upon wetting of the mutant site primers and the wild-type primers, heating the plurality of amplification sites and the plurality of detection sites to a temperature ranging from 60° C. to 65° C.;
(vi) detecting signals for turbidity and conductivity, via a charge-coupled device; and
(vii) determining, via the processor, based on the detected signals, whether a mutant is present in at least one of the plurality of detection sites and identifying, via the processor, a type of the mutant in at least one of the plurality of amplification sites:
if the processor determines that the virus or the bacterium is not present in the fourth well,
(i) adding DNA amplification buffer in the eighth well; and
(ii) determining, via the processor, whether a virus or a bacterium is present in the eighth well based on either fluorescence of SYBER Green dye or affinity probes.

19. The method of claim 17, the method further comprising:
performing the steps of claim 17 during a time period ranging from 5 to 30 minutes after an initial collection time associated with collecting the virus or bacterium that is airborne, via the air-handling and precipitation chamber, wherein the air-handling and precipitation chamber is configured for droplet deposition conducted via filtration, wherein the air-handling and precipitation chamber comprises a mechanical iris configured to move between an open position and a closed position, wherein the mechanical iris is configured such that a paper filter is disposed in an opening of the mechanical iris in the open position, and wherein the paper filter is configured to be crushed when the mechanical iris moves to the closed position such that the crushed paper filter is sized to be received in one of the plurality of well;
adding DNA amplification buffer in the eighth well; and
determining, via the processor, whether a virus or a bacterium is present in the eighth well via either fluorescence of SYBER Green dye or affinity probes.

20. The method of claim 15, further comprising:
rotating, via the second actuator, the reagent and reaction chamber over the second well after transferring the magnetic beads coupled to the vRNA or the pathogen DNA to the second well;
rotating, via the second actuator, the reagent and reaction chamber over the third well after transferring the magnetic beads coupled to the vRNA or the pathogen DNA to the third well; and
rotating, via the second actuator, the detection chamber over the fourth well after transferring the magnetic beads coupled to the vRNA or the pathogen DNA to the fourth well.

21. (canceled)

22. (canceled)

23. (canceled)

24. (canceled)

25. (canceled)

26. (canceled)
